(19) United States
(12) Patent Application Publication
(10) Pub. No.: US 2017/0290343 A1
(43) Pub. Date: Oct. 12, 2017

(54) ODOR MASKING AND ELIMINATING COMPOSITION USED BY HUNTERS

(71) Applicant: TIMOTHY ACOSTA, HIALEAH, FL (US)

(72) Inventor: TIMOTHY ACOSTA, HIALEAH, FL (US)

(21) Appl. No.: 15/094,028

(22) Filed: Apr. 8, 2016

(51) Int. Cl. A01N 65/44; A61Q 13/00; A61K 8/97; A61K 8/58

(52) U.S. Cl. CPC A01N 65/44; A61K 8/585; A61Q 13/00; A61K 8/97; A61K 2800/592

(57) ABSTRACT

A odor masking and eliminating composition that is used by sportsmen to hide their scent from prey. The odor masking and eliminating composition being made or composed of a cyclomethicone that is from about 80.0 percent to about 90.0 percent of the composition; a masking scent composition that is from about 5.0 percent to about 12.0 percent of the composition; an insect repellant composition that is from about 1.5 percent to about 2.0 percent of the composition; an odor eliminator that is from about 1.0 to about 3.0 percent of the composition; and an odor modification complex that is from about 2.0 to about 4.0 percent of the composition.

### BACKGROUND

The present invention is directed to an odor masking and eliminating composition that is used by sportsmen while hunting. The present invention can be applied to animate and inanimate objects and can also serve as an insect repellent.

The inventor of the present invention is an avid hunter whom understands the need to mask his scent when hunting.

Being a hunter, he knows that products have been developed to spray or apply to inanimate objects to mask the scent of a hunter. Yet, he has never found a product that could be safely applied to his physical being that would serve to mask his scent and eliminate the natural odors created by his body prior and during a hunting experience.

In addition, the inventor of the present invention realized that when he went hunting, often, he had to apply insect repellant in order to be able to effectively enjoy his hunting experience.

The need to mask and eliminate his odors while hunting while simultaneously allowing himself to be protected from insects is what motivated him to create the present composition for masking and eliminating odors that simultaneously serves as an insect repellent.

For the foregoing reasons, there is a need of an odor masking and eliminating composition that can be used by sportsmen or hunters on animate and inanimate objects.

### SUMMARY

The present invention describes an odor masking and eliminating composition that can be used by sportsmen or hunters on animate and inanimate objects. The composition can also serve as an insect repellent.

The present invention is an odor masking and eliminating composition that is used by sportsmen to hide their scent from prey. The odor masking and eliminating composition comprises of a cyclomethicone that is from about 80.0 percent to about 90.0 percent of the composition; a masking scent composition that is from about 5.0 percent to about 12.0 percent of the composition; an insect repellant composition that is from about 1.5 percent to about 2.0 percent of the composition; an odor eliminator that is from about 1.0 to about 3.0 percent of the composition; and an odor modification complex that is from about 2.0 to about 4.0 percent of the composition.

In a preferred embodiment, the present invention would be delivered in a spray fashion, yet other delivery methods are contemplated.

The Cyclomethicone used in the present invention is a class of a liquid silicone that possesses the characteristic of being of low viscosity and high volatility. It is also a skin emollient that can be used as a cleaning solvent. Cyclomethicones are cyclic siloxanes that are polymers that feature a monomer backbone of one silicon and two oxygen atoms that are bonded together, they have short backbones that make closed or nearly closed rings with their methyl groups. They are more volatile than dimethicones, so they are ideal for cosmetic products. The reason being that complete evaporation of the siloxanes eventually occurs and that is what makes them an excellent delivery compound for the present invention.

The masking scent of the present composition may be a synthetic fragrance or an essential oil. In a preferred embodiment, the essential oil would be either a pine or cedar oil. Synthetic fragrances used in the present invention would mimic smell or odors that one would normally encounter in outdoor environments in which one chooses to hunt.

In a preferred embodiment, the insect repellent used would be a citronella essential oil. It is foreseen that other similar repellents may be used in the present composition, so long as they would not be toxic or allergens.

In a preferred embodiment, the odor eliminator is a product that is sold under the brand ORDENONE that is manufactured by Belle-Aire Fragrances. It is an odorless product. It works by surrounding odor causing molecules and thereby eliminating malodor problems.

In a preferred embodiment, the odor modification complex is a compound that is also manufactured or sold by Belle-Aire Fragrances that is used to eliminate natural body odors, e.g., underarm and foot odors.

An object of the present invention is to provide a composition that can be used to aid hunters mask their scent.

Another object of the present invention is to provide a composition that can be used to eliminate body odors.

A further object of the present invention is to provide a composition that can be used as an insect repellant.

Yet a further object of the present invention is to provide a composition that can be used on animate and inanimate objects to hide the scent of the wearer when hunting.

### WRITTEN DESCRIPTION

The present invention is an odor masking and eliminating composition used for hunting. The composition comprises: a cyclomethicone that is from about 80.0 percent to about 90.0 percent of the odor masking and eliminating composition; a masking scent composition that is from about 5.0 percent to about 12.0 percent of the composition; an insect repellant composition that is from about 1.5 percent to about 2.0 percent of the composition; an odor eliminator that is from about 1.0 to about 3.0 percent of the composition; and an odor modification complex that is from about 2.0 to about 4.0 percent of the composition.

In a preferred embodiment, the masking scent composition is a synthetic fragrance that is 9.0 percent of the composition.

In another preferred embodiment, the masking scent is an essential oil that is 9.0 percent of the composition.

In still another preferred embodiment, the insect repellant is a citronella essential oil that is 2.0 percent of the composition.

In yet still another preferred embodiment, the odor eliminator is ORDENONE and it is 1.0 percent of the composition.

In a further preferred embodiment, the odor modification complex is a fragrance.

In still a further preferred embodiment, the cyclomethicone is 85.0 percent of the composition, the synthetic fragrance is 9.0 percent of the composition, the insect repellant is 2.0 percent of the composition, the odor eliminator is 1.0 percent of the composition, and the odor modification complex is 2.0 percent of the composition.

An advantage of the present invention is that it provides a composition that is used to aid hunters mask their scent.

Another advantage of the present invention is that it provides a composition that is used to eliminate body odors.

A further advantage of the present invention is that it provides a composition that is used as an insect repellant.

Yet a further object of the present invention is that it provides a composition that can be used on animate and inanimate objects to hide the scent of the user when hunting.

While the above description contains many specificities, these should not be construed as limitations on the scope, but rather as an exemplification of several preferred embodiments thereof. Many other variations are possible. Accordingly, the scope should be determined not by the embodiments illustrated, but by the appended claims and their legal equivalents.

What is claimed is:

1. An odor masking and eliminating composition used for hunting, comprising:
a cyclomethicone that is from about 80.0 percent to about 90.0 percent of the odor masking and eliminating composition;
a masking scent composition that is from about 5.0 percent to about 12.0 percent of the composition;
an insect repellant composition that is from about 1.5 percent to about 2.0 percent of the composition;
an odor eliminator that is from about 1.0 to about 3.0 percent of the composition; and
an odor modification complex that is from about 2.0 to about 4.0 percent of the composition.

2. The odor masking and elimination composition of claim 1, wherein the masking scent composition is a synthetic fragrance.

3. The odor masking and elimination composition of claim 2, wherein the insect repellant is a citronella essential oil.

4. The odor masking and elimination composition of claim 3, wherein the odor eliminator is ORDENONE.

5. The odor masking and elimination composition of claim 4, wherein the odor modification complex is a fragrance.

6. The odor masking fragrance of claim 5, wherein the cyclomethicone is 85.0 percent of the composition, the synthetic fragrance is 9.0 percent of the composition, the insect repellant is 2.0 percent of the composition, the odor eliminator is 1.0 percent of the composition, and the odor modification complex is 2.0 percent of the composition.

7. The odor masking and elimination composition of claim 1, wherein the masking scent composition is an essential oil.

8. The odor masking and elimination composition of claim 7, wherein the insect repellant is a citronella essential oil.

9. The odor masking and elimination composition of claim 8, wherein the odor eliminator is ORDENONE.

10. The odor masking and elimination composition of claim 9, wherein the odor modification complex is a fragrance.

11. The odor masking fragrance of claim 10, wherein the cyclomethicone is 85.0 percent of the composition, the essential oil is 9.0 percent of the composition, the insect repellant is 2.0 percent of the composition, the odor eliminator is 1.0 percent of the composition, and the odor modification complex is 2.0 percent of the composition.
